(19) United States
(12) Patent Application Publication
(10) Pub. No.: US 2003/0087830 A1
(43) Pub. Date: May 8, 2003

(54) Low molecular weight components of cartilage, complexes of metals with amino acids, DI-peptides and analogs thereof; processes for preparation and therapeutic uses thereof

(76) Inventors: Eric Dupont, Ste-Petronille, Ile d'Orleans (CA); Denis Lessard, Levis (CA); Serge Auger, St-Lambert (CA); Violetta Dimitriadou, Cap Rouge (CA); Pierre Falardeau, Sillery (CA); Patrick Poyet, St-Nicolas (CA)

Correspondence Address: HAYNES AND BOONE, LLP, 901 MAIN STREET, SUITE 3100, DALLAS, TX, 75202, US

(21) Appl. No.: 09/879,660

(22) Filed: Jun. 12, 2001

(51) Int. Cl. A61K 38/05; C07F 1/08

(52) U.S. Cl. 514/19; 548/402; 556/116

(57) ABSTRACT

Low molecular weight components extracted from shark cartilage and complexes made of copper with amino acid or dipeptide units or analogs thereof are disclosed. Methods are disclosed for the inhibition of angiogenesis (neovascularization) in an animal through the administration of these complexes, which results in treating angiogenesis-dependent diseases.

### FIELD OF THE INVENTION

The present invention relates to low molecular weight components obtainable from cartilage. The low molecular weight components exhibit antiangiogenic activity. More particularly, the invention relates to complexes made of amino acid, or dipeptide units, or naturally occurring and synthetic analogs thereof and copper. The invention further relates to the inhibition of angiogenesis (neovascularization) in an animal through the administration of one or more of these complexes, which results in treating angiogenesis-dependent diseases.

### BACKGROUND OF THE INVENTION

Processes for the preparation of cartilage extracts and the extracts themselves are disclosed in International Publication Nos. WO 95/32722, WO 96/23512 and WO 97/16197. Liquid extracts of cartilage have been tested in various assays for antiangiogenic, anticollagenolytic, direct anti-tumor proliferating and anti-inflammatory activities.

International Publication No. WO 95/32722 discloses a process for obtaining a shark cartilage extract having antiangiogenic, in vitro direct anti-tumor proliferating and in vivo anti-tumor activities. The process includes the steps of blending shark cartilage tissue and reducing the same to a particle size of about 500 μm in water; extracting active components into the water; and fractionating the extracts so obtained in order to recover molecules having molecular weights less than about 500 kDa (0-500 fraction). The liquid cartilage extract was concentrated on a membrane having a nominal porosity of about 1 kDa to form a concentrated liquid extract comprising molecules having molecular weights less than about 500 kDa. The extract was enriched in molecules having molecular weights between about 1-500 kDa. The 0-500 fraction was further fractionated to form a plurality of extracts containing anti-tumor proliferating molecules having molecular weights ranging from about 1 to 120 kDa.

International Publication No. WO 96/23512 discloses a process for extracting biologically active components from a source of cartilage in aqueous solutions. This publication also discloses other biological activities associated with liquid shark cartilage, namely anticollagenolytic and anti-inflammatory activities.

International Publication No. WO 97/16197 discloses a process for the recovery of an aqueous extract enriched in molecules having molecular weights between about 0.1 to 500 kDa.

U.S. Pat. No. 6,168,807 discloses a process by which organic solvent-containing solutions are used in lieu of pure water for the preparation of cartilage extracts. This patent also discloses a process by which a total liquid extract of shark cartilage composed of molecules having molecular weights less than about 500 kDa (0-500 fraction) is purified into two well separated fractions composed of molecules having molecular weights less than about 1 kDa (0-1 fraction) and between about 1-500 kDa (1-500 kDa fraction). This patent further discloses a component which has a mass of 244 amu (atomic mass unit) and which is capable of gelatinase inhibition.

Thus, it is disclosed in the art how to obtain and use shark cartilage extracts having anti-angiogenic, anti-tumor, anti-collagenolytic and anti-inflammatory activities.

U.S. Pat. No. 5,902,790 discloses a composition containing thymogen-like molecules that are di-peptides or longer peptides comprising glutamyl-tryptophan having angiostatic properties in an ex ovo assay (CAM).

Amino acids and peptide mixtures containing divalent metals are also disclosed in the art. They were tested in a form of a metal proteinate to increase metal concentrations in biological tissues in animals and plants.

U.S. Pat. No. 4,020,158 discloses a method of raising the level of essential bivalent metals in the tissues of animals, which comprises administering a metal proteinate to the animal. The metal proteinate is comprised of one or more protein hydrosylates selected from a group consisting of polypeptides, peptides, and amino acids, chelated with a metal.

U.S. Pat. Nos. 4,599,152 and 4,830,716 disclose methods for the preparation of amino acid chelates that are essentially free of interfering anions.

U.S. Pat. No. 4,863,898 discloses amino acid metal chelate compositions, which are formulated for delivery to one or more specific tissues within a living organism.

U.S. Pat. No. 5,162,369 discloses a composition and a method of enhancing the immune system of a warm-blooded animal afflicted with a form of antigenic morbidity.

Creatine (also known as N-(Aminoiminomethyl)-N-methylglycine, methylglycoamine or N-methyl-guanido acetic acid) is a naturally occurring nitrogenous compound found in mammalian skeletal muscle, brain, and other organs. Cohn (U.S. Pat. No. 5,576,316) and Kaddurah-Daouk et al. (International Publication Nos. WO 92/08456 and WO 92/09192) describe methods for inhibiting tumor growth rate using creatine or creatine analogs. Wheelwright (U.S. Pat. No. 6,114,379) describes methods to protect creatine from undergoing cyclization in the acidic environment of the stomach by using creatine-metal complex. This patent also describes a method to make a metal more bioavailable due to the presence of the creatine ligand.

Copper-peptides, including the Glycyl-histidyl-lysine : Copper (II) complex, are able to accelerate the regeneration and repair of many types of mammalian tissue (Maquart et al., In vivo stimulation of connective tissue accumulation by the tripeptide-copper complex glycyl-histidyl-lysine-Cu(II) in rats experimental wounds, J. Clin. Invest. 92:2368-76,1993; U.S. Pat. No. 5,164,367; and U.S. Pat. No. 5,382,431). Such peptide complexes are angiogenic (Sage and Vernon, Regulation of angiogenesis by extracellular matrix: the growth and the glue, J. Hypertension Supplement 12(10):S145-152, 1994; Raju et al., Ceruloplasmin, copper, and angiogenesis JNCI, 69:1183-1188, 1982). Further, such peptide complexes are able to activate matrix metalloproteinases (MMPs; Simeon et al., Expression and activation of matrix metalloproteinases in wounds: modulation by the tripeptide-copper complex glycyl-histidyl-lysine-Cu(II J. Invest Dermatol. 112 957-64, 1999). Accordingly, it is known in the art that some specific tripeptide complexes with copper increase angiogenesis.

Copper has been shown to play a prime importance in angiogenesis, since a copper deficient rabbit cannot induce angiogenesis (Raju et al. Ceruloplasmin, copper, and angiogenesis JNCI, 69:1183-1188, 1982). This is supported by other results showing that the angiogenic activity of four angiogenic cytokines (IL-1, bFGF, TNF-alpha and VEGF) is copper dependant (Brem S. Angiogenesis and cancer control: from concept to therapeutic trial, Cancer Control. 6:436-458, 1999; Hu G F. Copper stimulates proliferation of human endothelial cells under culture. Cell Biochem 69:326-335, 1998). Moreover, copper reduction obtained through administration of a low-copper diet and a chelator of copper inhibits angiogenesis in the animal while copper repletion restores angiogenesis. Chelators of copper include penicillamine, tetrathiomolybdate and captotril.

Angiogenesis is defined as the formation of new blood vessels from pre-existing capillaries. Almost all tissues and organs develop a vascular network, which provides cells with nutrients and oxygen and enables the elimination of metabolic wastes. Once formed, the vascular network is a stable system that regenerates slowly. It is essential to embryonic development. In adults, angiogenesis is a limited process which occurs primarily during wound healing and the female reproductive cycle (ovulation, menstruation, implantation, and pregnancy). It also plays a critical role in the pathophysiology of approximately 20 diseases classified as angiogenesis-dependent. Excessive angiogenesis has been observed in several pathologies including cancers (both solid and hematologic tumors), chronic inflammation (rheumatoid arthritis, Crohn's disease), psoriasis, scleroderma, rosacea, hemangioma, hypertrophic scarring and other skin diseases (Sauder and Thibodeau, Angiogenesis in Dermatology, Curr. Probl. Dermatol, May/June 2001, in press), endometriosis, adiposity, diabetic retinopathy, neovascular glaucoma, macular degeneration, ocular herpes, trachoma and corneal graft neovascularization and other ocular vascular diseases, and cardiovascular diseases (atherosclerosis) (Griffloen A W. Angiogenesis: Potentials for Pharmacologic Intervention in the Treatment of Cancer, Cardiovascular Diseases, and Chronic Inflammation, Pharmacol. Rev. 52:237-68, 2000).

Angiogenesis requires the cooperation of a variety of molecules that regulate cellular processes such as extracellular matrix (ECM) remodeling, invasion, migration and proliferation. It can be organized into three major phases: an initiation phase, a proliferative/invasive phase and a differentiation/maturation phase. The initiation phase can be triggered by activation of vascular cells via a variety of angiogenic cytokines and other physiological mediators. The proliferation/invasion phase of angiogenesis is characterized by endothelial cell replication, re-organization of the cytoskeleton and of proteins involved in membrane adhesion, and production of proteases that are secreted to promote endothelial cell migration in the surrounding matrix. Finally, the differentiation/maturation phase is characterized by endothelial cell production of a basement membrane, lumen formation and junctional coupling with other cells.

There is increasing evidence suggesting that chronic inflammation and angiogenesis are closely dependent. Because angiogenesis and inflammation may be encountered alone or in combination in a large variety of diseases or conditions, a product capable of antagonizing at least these activities without affecting normal body functions would be of a great therapeutic value.

Given the interest in components obtained from shark cartilage because of their efficacy and inocuousness, there exists the need for providing therapeutic compounds isolated or derived therefrom.

### SUMMARY OF THE INVENTION

According to the present invention, complexes that include two amino acids bound to a copper ion, and having anti-angiogenic activity, have been isolated from shark cartilage extracts. Various structures sharing this general formula have been designed, synthesized, and tested for the same potentially useful activity.

According to a preferred embodiment of the present invention, an anti-angiogenic compound is provided that includes two units complexed to a copper metal ion, wherein the units are independently selected from an amino acid, a dipeptide or an analog thereof which has a carboxyl and an amino group capable of complexing with copper and that targets cells of an angiogenic tissue. Preferably, the amino acids are selected from threonine, aspartic acid, glutamic acid, glycine, alanine, valine, leucine, isoleucine, arginine, lysine, proline, glutamine, serine and histidine. In another preferred embodiment of the present invention, the dipeptide is glutamyl-tryptophane. In yet another preferred embodiment of the present invention, the analog is creatine or a creatine-derivative.

According to still another preferred embodiment of the present invention, a composition of matter is provided that includes an effective amount of the anti-angiogenic compounds of the present invention and a pharmaceutically acceptable vehicle. According to another preferred embodiment of the compositions of the present invention, the amino acid is selected from the group consisting of threonine, aspartic acid, glutamic acid, glycine, alanine, valine, leucine, isoleucine, arginine, lysine, proline, glutamine, serine, histidine, or any mixture thereof. According to another preferred embodiment of the compositions of the present invention, the composition further includes an anti-inflammatory, an anti-tumor agent, an anti-oxidant, or an anti-collagenolytic agent. According to still another preferred embodiment of the compositions of the present invention, the anti-tumor agent comprises a shark cartilage extract. According to still another preferred embodiment of the compositions of the present invention, the anti-tumor agent comprises a shark cartilage extract and an anti-neoplastic agent.

The present invention is also directed to a method for selectively inhibiting angiogenesis which comprises the step of administering to a subject an effective amount of an anti-angiogenic compound according to the present invention.

The present invention is further directed to a method for obtaining a compound which has an anti-angiogenic activity from a cartilage material which comprises the steps of:

a) extracting the activity from cartilage material reduced to solid particles whose size is lower than or equal to about 500 μm into an aqueous solution, resulting in a homogenous mixture of the solid particles and a first liquid extract having the activity;

b) separating solid particles from the first liquid extract;

c) fractionating the first liquid extract, to recover a second extract comprising molecules having a molecular weight lower than about 500 KDa; and

d) treating the second extract under denaturating conditions of pH or temperature to generate a low molecular weight compound comprising amino acids, dipeptides, analogs thereof, and copper-complexes thereof. According to a further preferred embodiment of the methods of the present invention, the pH ranges from about 2 to about 6.

According to another further preferred embodiment of the methods of the present invention the temperature is of about 37° C. to 100° C. According to still another further preferred embodiment of the methods of the present invention the method further comprises the step of purifying the compound from the neutralized extract.

The present invention is also directed to cartilage extracts obtained from the processes of the present invention.

The following embodiments and figures are part of the present specification and are included to further demonstrate certain aspects of the invention. The invention may be better understood by reference to one or more of these figures in combination with the detailed description of the preferred embodiments presented herein, which do not have the purpose of limiting the scope of the invention.

### BRIEF DESCRIPTION OF THE DRAWINGS

FIG. 1 represents the general procedure used to purify low molecular weight inhibitors of angiogenesis from water soluble shark cartilage extract.

FIG. 2 depicts a representative UV:254 nm (A) and total ions (B) chromatograms of the SPE-DIOL-E-(2) fraction, injected on the LC/MS/MS system equipped with an analytical HPLC-diol column operating in a linear gradient from (5% ammonium formate (5 mM pH 3)/95% methanol to 95% ammonium formate/5% methanol in 15 minutes at a flow of 1 ml/min).

FIG. 3 depicts the mass spectra of the fourteen minute peak (AE-994) extracted from the chromatogram shown in FIG. 2.

FIG. 4 depicts a representative UV:254 nm (A) and total ions (B) chromatograms of the (Asp)₂-Cu complex (Compound No. 6) injected on the LC/MS/MS system equipped with an analytical HPLC-diol column operating in a linear gradient from (5% ammonium formate (5 mM pH 3)/95% methanol to 95% ammonium formate/5% methanol in 15 minutes at a flow of 1 ml/min).

FIG. 5 depicts the mass spectra of the fourteen minute peak the (Asp)₂-Cu complex (Compound No. 6) extracted from the chromatogram shown in FIG. 4.

FIG. 6 shows the effect of test samples on ex vivo angiogenesis in chick embryos.

FIG. 7 shows the effect of test samples on tubulogenesis.

FIG. 8 shows the antitumoral activity of a composition consisting of a mixture of 8 amino acid copper complexes on experimental glioblastoma in nude mice.

FIG. 9 shows the additive effect of a composition consisting of a mixture of 5 amino acid copper complexes added to a soluble cartilage extract on endothelial cell proliferation.

### DETAILED DESCRIPTION OF THE INVENTION

According to the processes of the present invention, various fractions of cartilage are prepared in the following manner.

The 0-500 fraction: The 0-500 fraction is a shark cartilage liquid extract comprising components having molecular weights less than about 500 kDa. Preparative methods for the 0-500 fraction are disclosed in International Publication Nos. WO 95/32722, WO 96/23512, and WO 97/16197, the entire disclosures of which are hereby incorporated herein by reference. These methods comprise the steps of:

homogenizing shark cartilage in an aqueous solution in conditions compatible with the preservation of the integrity of biologically active components present in cartilage until the cartilage is reduced to solid particles whose size is less than about 500 μm;

extracting the biologically active components into the aqueous solution, which results in a mixture of solid particles and of crude liquid extract (LE) having the biologically active components;

separating the liquid extract from the solid particles;

further separating the crude liquid extract so as to obtain a final liquid extract containing molecules having molecular weights less than about 500 kDa (LE-0-500); and

filtering the LE-0-500 on a microfiltration membrane (0.22 micron) and freezing to obtain the final liquid extract (0-500 fraction).

The 0-1 and 1-500 fractions: The 0-1 fraction is a shark cartilage liquid extract comprising components having molecular weights less than about 1 kDa. The 1-500 fraction is a shark cartilage liquid extract comprising components having molecular weights between about 1-500 kDa. Preparative methods for the 0-1 and 1-500 fractions are also disclosed in International Publication Nos. WO 95/32722, WO 96/23512, and WO 97/16197. These methods comprise the steps of:

filtering the LE-0-500 with a membrane having a nominal molecular weight cut-off of about 1 kDa to form permeate liquid extracts comprising cartilage molecules having molecular weights less than about 1 kDa (P 0-1), and retentate liquid extracts comprising cartilage molecules having molecular weights greater than about 1 kDa (R 0-1); and;

microfiltering the retentate and permeate liquid extracts through a microfiltration membrane having a porosity of about 0.22 microns.

Antiangiogenic activity was observed in the 0-1 fraction as well as the 1-500 fraction.

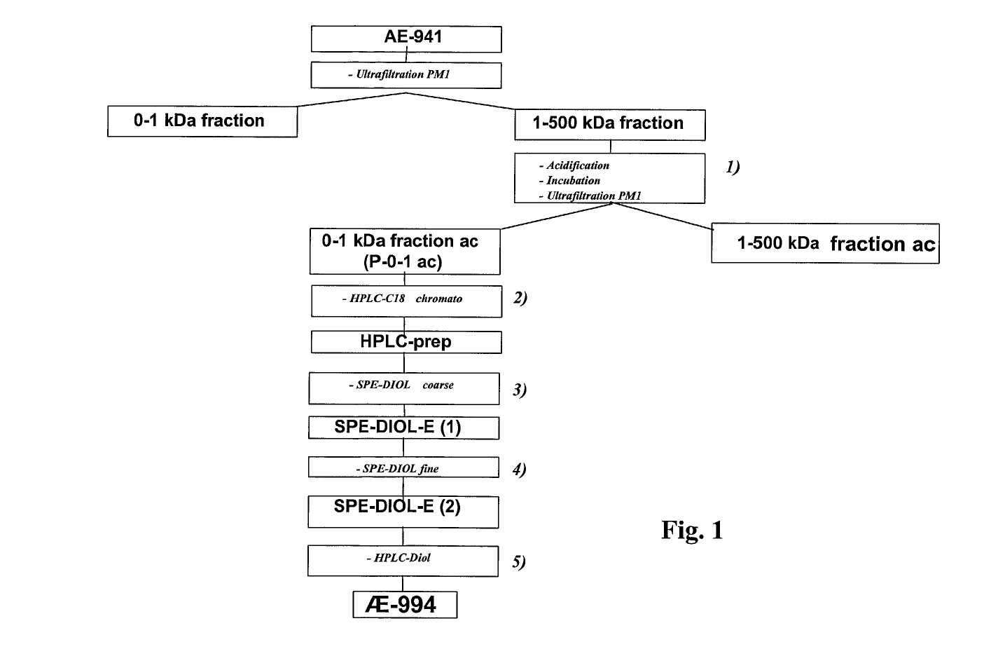

According to the processes of the present invention, the next step was to isolate active components from the 1-500 fraction (FIG. 1). The endothelial proliferation and the EVT assays discussed below were used to evaluate the biological activity present in each of the fractions obtained from the following procedure.

Everywhere a specific value is given in the present disclosure, such a value should be extended to cover a reasonable margin of error. Such extended language is intended by using either a value alone, or that value preceded by the term “about”. A margin of error should be acceptable to take into account the lack of perfect precision of an apparatus, a piece of equipment, or the evaluation of precision from a person performing an experiment, or even the principle of normal distribution under a curve. For example, a membrane having a nominal porosity of 1 or 500 KDa will always present a certain proportion of pore sizes larger or smaller than the indicated value. The molecules permeating such a membrane would therefore have a molecular weight located somewhere between about 0 and about 1 KDa or 500 KDa, respectively. Another example is the homogenization step which provides particles having an average size of about 500 μm. One of ordinary skill in the art will understand that larger and smaller particles can result from that step, following a normal distribution under a curve (size vs. speed or time of homogenization).

Preparation of an Active Fraction (P0-1AC) by Acidic Incubation and Ultrafiltration of the P-1-500 kDa Fraction

An initial volume of P-1-500 kDa fraction was acidified to pH 2.5 with concentrated trifluoacetic acid. The resulting solution was incubated at 37° C. for one hour. After cooling, this solution was ultrafiltrated, on a system equipped with a 1 kDa cut-off membrane (PM1, Koch membrane, #0720032). Three cycles, consisting of ultrafiltering the solution until its volume reached half of its initial volume, followed by water addition to restore the solution to its initial volume, were performed. The combined acidified fractions of P0-1 kDa were concentrated under reduced pressure until the final volume reached 1/100 of the initial P-1-500 kDa volume used. This final solution was labeled (P0-1AC) and kept frozen until used.

Purification of an Active Fraction (HPLC-C18 Prep) from the P0-1AC Fraction by Preparative High Pressure Liquid Chromatography (HPLC)

The (P0-1AC) fraction was further purified on a preparative HPLC system equipped with a Prep-C18 column (10 μm, 250×50 mm, Phenomenex; 00G-4088VO) and operated isocratically with a 0.1% trifluoroacetic acid in water mobile phase. After injection of 5 ml of the (P0-1AC) fraction on the chromatographic system, biological activity was observed in the fraction collected between 8 and 10 minutes. Collected fractions (8-10 minutes) from multiple injections were pooled and then evaporated to dryness. The final residue was made pH neutral when resolubilized in a 20% methanol/water solution by multiple cycles of solubilization and evaporation of this solution. The neutralized dried residue was labeled HPLC-C18 prep and kept frozen until used.

Coarse Purification of an Active Fraction (SPE-DIOL-E-(1)) from the HPLC-C18 Prep Fraction by Solid Phase Extraction (SPE)

The final dry residue HPLC-C18 prep was solubilized with a mixture of methanol: isopropanol (1:1) using a total volume corresponding to 1/1000 of the 1-500 kDa fraction used initially. A SPE-Diol column (3 cc, 500 mg; Supelco; 57016) was conditioned with 4 ml of methanol followed by 6 ml of ethyl acetate. Then 0.2 ml of the resolubilized HPLC-C18 prep fraction was mixed with 2.5 ml of ethyl acetate and transferred to the conditioned SPE-Diol column. The SPE-Diol column sorbent was then washed with 15 ml of isopropanol and the bioactive compounds were eluted with 5 ml of a solution containing 10 percent water in methanol. The collected active fraction (SPE-DIOL-E-(1)) was then evaporated to dryness and keep frozen until used.

Fine Purification of an Active Fraction (SPE-DIOL-E-(2)) from the (SPE-DIOL-E-(1) Fraction) by Solid Phase Extraction (SPE)

The final dry residue SPE-DIOL-E-(1) was solubilized with a mixture of methanol: isopropanol (1:1) using a total volume corresponding to 1/1000 of the 1-500 kDa fraction used initially. A SPE-Diol column (3 cc, 500 mg; Supelco 57016) was conditioned with 4 ml of methanol followed by 6 ml of ethyl acetate. Then 0.2 ml of the resolubilized SPE-DIOL-E-(1) fraction was mixed with 2.5 ml of ethyl acetate and transferred to the conditioned SPE-Diol column. The SPE-Diol column sorbent was then washed with 5 ml of isopropanol, 5 ml of an isopropanol:methanol (9:1) solution and 10 ml of an isopropanol:methanol (1:1) solution. The active compounds were eluted with 5 ml of a solution containing 10 percent water in methanol. The collected active fraction (SPE-DIOL-E-(2)) was then evaporated to dryness and kept frozen until used. (Chemical analysis of this residue by liquid chromatography/mass spectrometry (LC/MS), and inductively coupled plasma/mass spectroscopy (ICP/MS) (which techniques are well known to those of ordinary skill in the art) revealed the presence of Cu(II), Ala, Asp, Gln, Glu, Gly, Pro, Ser, and Thr).

Identification of an Active Component (AE-994) from the SPE-DIOL-E-(2) Fraction by High Pressure Liquid Chromatography/Mass Spectrometry (HPLC/MS).

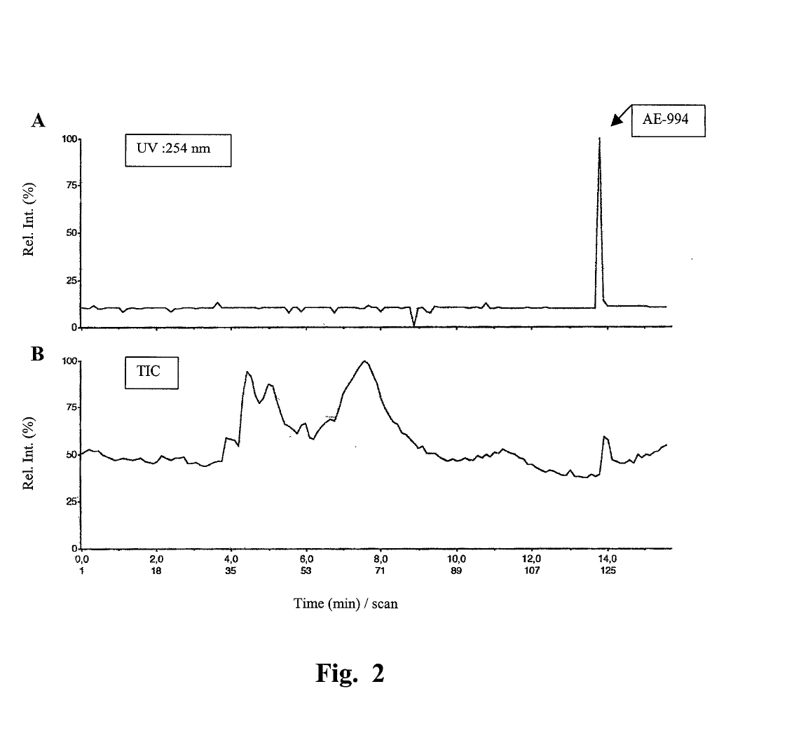

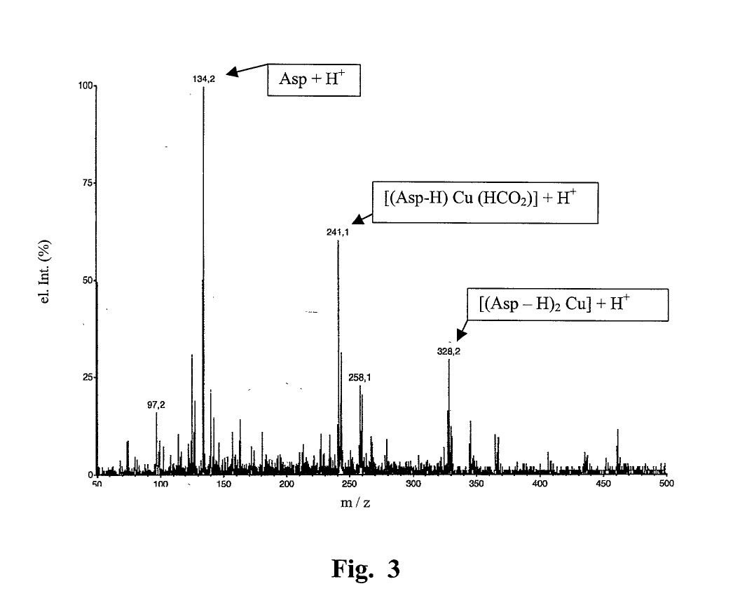

The active fraction SPE-DIOL-E-(2) was injected on a HPLC/MS/MS system (API III; Sciex). The HPLC was equipped with an analytical Diol column (5 μm, 250×4.6 mm, Supelco 58201) and operated in gradient mode, (linear gradient from 5% ammonium formate (5 mM pH 3)/95% methanol to 95% ammonium formate (5 mM pH 3)/5% methanol in 15 minutes). The mass spectrometer (MS) system was equipped with an Ions Spray source and operated in Q1 positive ions scanning mode. Biological activity from collected fractions, obtained by splitting the column effluent between the MS and a fraction collector, was observed under the peak eluting at 14.0 minutes (FIG. 2). The mass spectrum of this chromatographic peak showed ions corresponding to [(ASP−H)₂Cu]+H⁺ m/e 328 and 330 amu, [(ASP−H)Cu(HCO₂)]+H⁺ m/e 241 and 243 amu and ASP+H⁺ m/e 134 amu (FIG. 3).

The process of the present invention is directed to the preparation of fractions obtained from cartilage that possess antiangiogenic activity. The purification and identification of such fractions unexpectedly indicated that a complex of the amino acid aspartate bound with copper in a ratio of 2 molecules of aspartate to one molecule of copper (see compound 6 in Table II) possesses antiangiogenic activity. Since the SPE-Diol-E-(2) fraction comprises 8 amino acids and shows strong antiangiogenic activity, it is believed that all eight amino acids are capable of complexing with copper. Surprisingly, low molecular weight components (less than about 1 kDa), comprising copper amino acids and complexes thereof, having antiangiogenic activity were obtained after the acidification of the 1-500 kDa fraction and ultrafiltration. Moreover, treatment of the 1-500 kDa fraction with heat at 37° C. to 100° C. also generated low molecular weight complexes with copper(II). This indicates that cartilage extract contains high molecular weight precursors which can interact with copper (II). Such precursors could also have antiangiogenic activity. This step (acidification and/or heat) is preferred for the generation of the amino acid or dipeptide copper complexes from the extract. According to the present invention, the acidification step, preferably is conducted using trifluoroacetic acid, hydrochloric acid or sulfuric acid. Those of ordinary skill in the art will recognize that other suitable acids include acetic acid, formic acid, phosphoric acid, trichloroacetic acid and citric acid. Acids should be used in an amount providing a pH range of about 2 to 6. Therefore, it is believed that the present process includes a denaturation step, or a step achieving disturbance of chemical equilibrium, and/or a mild hydrolysis step, which detach small molecules from bigger ones which are precursors of the present compounds.

The biological properties of various compositions according to the present invention were determined by using at least one of the following assays:

Embryonic Vascularization Test (EVT): an assay for evaluating spontaneous antiangiogenic activity ex vivo;

Endothelial cell proliferation, migration and tubulogenesis; assays for evaluating antiangiogenic activity in vitro;

Matrigel™ in vivo: an assay for evaluating antiangiogenic activity in mice;

Human derived glioblastoma graft to nude mice model (C6): an assay for evaluating anti-tumor activity; and

Lewis Lung Carcinoma metastatic mouse model (LLC): an assay for evaluating anti-metastatic activity.

The Embryonic Vascularization Test (EVT) was performed to determine the ability of test samples to inhibit the formation of new blood vessels (antiangiogenic activity).

The normal development of a chick embryo involves the formation of an external vascular system located in the vitelline membrane which carries nutrients from the vitellus (yolk) to the developing embryo. When placed onto the vitelline membrane, antiangiogenic substances can inhibit the blood vessel formation that occurs in the vitelline membrane. Briefly, methylcellulose discs (an inert solid and transparent matrix) containing different test samples were placed on the external border of the vascular perimeter of the vitelline membrane, where the angiogenic process occurs. Vascularization was assessed 24 hours after disc deposition, and results were expressed as the percent of embryos in which blood vessel formation was affected. The blood vessel formation was considered affected when its growing path was either deviated, or diminished or when there was no growth observed beyond the disc as compared to the negative control. The results are expressed as 0, +, and ++ where 0 is for a lower response than 25 percent of the eggs; + is for a response between 25 and 50 percent; and ++ is for an antiangiogenic response observed in more than 50 percent of the eggs.

Endothelial and tumor cell proliferation assays were performed to determine the ability of test samples to inhibit endothelial cell proliferation (antiangiogenic activity), normal cell proliferation,and tumor cell proliferation.

Endothelial cells (Human umbilical vein endothelial cells (HUVEC), bovine aortic endothelial cells (BAEC)), skeletal muscle cells, human dermal fibroblasts (all from Clonetics, Walkersville, Md.) were respectively maintained in EBM-2, MDEM, SKGM and FGFM-2 complete media according to manufacturer's instructions. Tumor rat glioblastoma cell line (C6) was maintained in DMEM (Sigma, Oaksville, Ontario) supplemented with 10% fetal calf serum (FCS) and antibiotics in 5% CO₂. Briefly, cells seeded in 96-well sterile tissue culture dishes were treated with increasing concentrations of different test samples for a period of 48 hours at 37° C. Cell number was then evaluated by a calorimetric assay using the cell proliferation reagent tetrazolium salt and the fluorimetric assay using DNA dye (Hoescht) according to the procedure established by the manufacturer (Boehringer Mannhein). The conversion of these tetrazolium salts into formazan occurs only in metabolically active cells. The percentage of cell inhibition was determined by comparison to untreated cells. Results, expressed as EC₅₀ (the concentration of compounds needed to reduce by 50 percent the cell number as compared to the control untreated cells), correspond to the average of at least three independent experiments.

The endothelial cell migration assay was performed to determine the ability of test samples to inhibit the chemotaxic activity of vascular endothelial growth factor (VEGF;R/D System, MN) using a modified Boyden chamber (Transwell™, Corning/Costar). Briefly, polyvinylidene difluoride filters (PVDF) were coated with Pronectin F (Biosources, CA), a constituent of basement membrane barrier. Boyden chambers were assembled by adding VEGF to the culture media in the lower chamber (chemoattractant). Endothelial cells suspended in cultured media in the presence or the absence of the test samples were added to the upper chamber. The chambers were then incubated for a period of 4.5 hours at 37° C. At the end of the incubation period, the cells from both side of the PVDF membrane were fixed with 3.7% formaldehyde. Cells from the upper surface of the membrane were mechanically removed, and the cells remaining on the underside of the membrane were stained with crystal violet (0.5%; Sigma). Cells were then carefully rinsed and the dye fixed to the cells was quantified with a microplate reader (590 mm) after its solubilization with a solution of 50% citric acid (0.1M) in water and 50% ethanol.

The endothelial cell tubule formation assay is a semi-quantitative assay that was performed to determine the ability of test samples to inhibit this multi-step process, which involves cell adhesion, migration, differentiation and growth, using a commercial kit (Chemicon). Briefly, endothelial cells were seeded on solid gel of Ecmatrix™ (Chemicon), basement proteins prepared from the Engelbreth Holm-Swarm (EHS) mouse tumor in microplate in a E-99 media (Sigma) supplemented with 5% FBS and VEGF (50 ng/ml) in the presence or absence of the test sample. Cells were incubated for a period of 24 hours at 37° C. Pictures were taken on the entire tissue culture dish using a binocular microscope for further evaluation.

The Matrigel™ in vivo assay was performed to determine the ability of test samples to inhibit blood vessel formation (antiangiogenic activity) and to establish their bioavailability in vivo.

The Matrigel™ assay was performed as previously described (Kerr, J. S., et al. M. Novel small molecule av integrin antagonists: comparative anti-cancer efficacy with known angiogenesis inhibitors, Anticancer Research 19: 959-968, 1999). Briefly, liquid Matrigel™ (Becton Dickinson, Bedford, Mass.) was maintained at 4° C. and mixed thoroughly for at least 3 hours with 1,500 ng/ml basic fibroblast growth factor (bFGF) (R&D Systems, Minneapolis, Minn.) used as the primary angiogenic stimulus. The mixture (0.5 ml) was injected subcutaneously into the ventral midline of C57B1/6 mice, and treatment with different test samples or saline solution was initiated the same day. Additional animals were also injected with Matrigel™ containing no bFGF to serve as baseline controls. The animals were treated daily for a 10 day period (intra-peritonealy, 0.2 ml of each dose). At day 11, animals were injected with FITC-dextran 20 min prior to euthanizing treated mice. Matrigel™ gels were removed along with underlying peritoneum and prepared for FITC-dextran detection by fluorimetry. The concentration of FITC-dextran in the gels was calculated from a known concentration of FITC-dextran using a standard curve. The FITC-dextran content of the gels from compound-treated animals and controls was expressed as a percent of the positive bFGF-treated controls after the baseline control of FITC-dextran levels were subtracted from all groups.

The rat glioblastoma derived cell (C6) grafted to nude mice was used to determine the ability of different test samples to inhibit the formation of a primary tumor (anti-tumor activity) as well as to inhibit tumor angiogenesis. Briefly, C6 cells were transplanted subcutaneously in the exterior part of the posterior leg of nude mice. Treatment with test samples started 3 days before inoculation. Mice were treated daily by intra-peritoneal injection (0.2 ml of each dose) for 21 days following inoculation. Tumor volume was evaluated every 3 days. At day 25, animals were sacrificed and tumors were prepared for evaluating vessel density by immunochemistry (factor VIII). Factor VIII is a good marker for the detection of blood vessels.

The Lewis Lung Carcinoma mouse model (LLC) was used to determine the ability of different test samples to inhibit the formation of metastases within the lung. The Lewis lung carcinoma clone M27, with a high metastatic potential to the lung, was established by Dr P. Brodt (Brodt P, Characterization of two highly metastatic variants of Lewis lung carcinoma with different organ specificities. Cancer Res., 46: 2442, 1986). This model is well established and is known for its predictive correlation between in vitro and in vivo activity. Briefly, LLC cells were transplanted subcutaneously in the axillary region of the right flank of C57BL/10 female mice (Charles River Inc.) When the primary tumor reached a size of 0.5-1.0 cm³ (day 10 post-inoculation), the tumor was carefully separated from the surrounding healthy tissues. Treatment with different test samples started the day following tumor removal (day 11 post-inoculation). Saline or different test samples were administered daily for two weeks by intraperitoneal injection (0.2 ml of each dose). As previous experiments had shown that a period of approximately two weeks after removal of the primary tumor was sufficient to obtain an average of 30 to 50 nodules on the lung surface, animals were sacrificed in a CO₂ chamber two weeks later. Following autopsy, both lungs were removed, weighed and fixed in 10% Bouin's fixative. Lung surface metastases were counted using a stereomicroscope (4×).

The biological activity of the (AsP)₂-CU (compound No. 6; -AE-994) was evaluated on various systems with respect to its antiangiogenic activity. As represented in Table I, AE-994 (compound No. 6) showed good biological activities. On the other hand, copper salt and free-aspartate did not show any significant activity in these particular assays (data not shown).

TABLE I
Antiangiogenic activity of æ-994 (Compound 6)

| Assays | Activities |
|---|---|
| Antiangiogenic activity | |
| EVT (0.6 mg) | ±38% of inhibition |
| Cell proliferation | |
| Endothelial cells | *EC₅₀ = 0.29 mM |
| Fibroblasts | EC₅₀ = 1.15 mM |
| Striated muscle cells | Non-active |
| C6 cells | Non-active |
| Migration (1.25 mmole) | 82% of inhibition |
| Tubulogenesis (1.25 mmole) | Decrease of capillary-like tube formation |
| | and absence of pentagonal network |
| Matrigel in vivo | |
| 0.14 mg/Kg | 27.7% of inhibition |
| 1.14 mg/Kg | 22.6% of inhbition |
| 2.5 mg/Kg | 42% of inhibition |
| 7.0 mg/Kg | 56.8% of inhibition |

*EC₅₀; is the concentration of compound corresponding to 50% of the effect

All amounts are given in equivalent copper.

This surprising discovery led to an investigation to determine whether other amino acids/peptides, and molecules comprising amino and carboxylic groups that are able to be complexed with copper or other divalent metals in aqueous solution have a similar biological activity. Toward this end, several analogs were prepared using the following general procedure (Table II, Table III, and Table IV).

TABLE II
Description of analogs

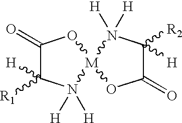

| | | | Metal | Complexes | Affinity of the |
|---|---|---|---|---|---|
| No.* | R1 | R2 | (M) | AA:Cu | AA with Cooper |
| 1 | —H | —H | Cu | Gly:Cu:GIy | 16.0 |
| 2 | —CH₃ | —CH₃ | Cu | L-Ala:Cu:L-Ala | 15.9 |
| 3 | —CH₂OH | —CH₂OH | Cu | L-Ser:Cu:L-Ser | 15.4 |
| 4 | —CH(CH₃)₂ | —CH(CH₃)₂ | Cu | L-Val:Cu:L-Val | 15.2 |
| 5 | —CH(OH)CH₃ | —CH(OH)CH₃ | Cu | L-Thr:Cu:L-Thr | 15.4 |
| 6 | —CH₂COOH | —CH₂COOH | Cu | L-Asp Cu L-Asp | 16.0 |
| 7 | —CH₂CH₂COOH | —CH₂CH₂COOH | Cu | L-Glu:Cu:L-Glu | — |
| 8 | 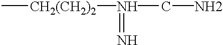 | 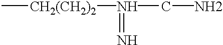 | Cu | L-Arg:Cu:L-Arg | 14.9 |
| 9 | —CH₂(CH₂)₃—NH₂ | —CH₂(CH₂)₃—NH₂ | Cu | L-Lys:Cu:L-Lys | 14.4 |
| 10 | 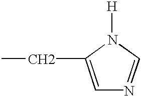 | 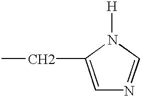 | Cu | L-His:Cu:L-His | 19.4 |
| 11 | —CH₂CH₂CONH₂ | —CH₂CH₂CONH₂ | Cu | L-Gln:Cu:L-Gln | 15.5 |
| 12 | —CH(CH₃)CH₂—CH₃ | —CH(CH₃)CH₂—CH₃ | Cu | L-Ile:Cu:L-IIe | 16.6 |

Examples of D-alpha-Amino Acids complexed with Cu (II)

| | | | | | |
|---|---|---|---|---|---|
| 13 | —CH₃ | —CH₃ | Cu | D-Ala:Cu:D-Ala | — |
| 14 | —CH₂COOH | —CH₂COOH | Cu | D-Asp:Cu:D-Asp | — |
| 15 | —CH₂CH₂COOH | —CH₂CH₂COOH | Cu | D-Glu:Cu:D-GIu | — |
| 16 | —CH(OH)CH₃ | —CH(OH)CH₃ | Cu | D-Thr:Cu:D-Thr | — |
| 17 | 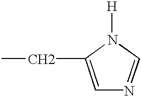 | 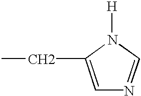 | Cu | D-His:Cu:D-His | — |

Examples of Hetero-alpha-Amino Acids complexed with Cu (II)

| | | | | | |
|---|---|---|---|---|---|
| 18 | —H | —CH₃ | Cu | L-Ala:Cu:Gly | — |
| 19 | —CH₃ | —CH(OH)CH₃ | Cu | L-Ala:Cu:L-Thr | — |
| 20 | —CH₃ | —CH₂CH₂COOH | Cu | L-Ala:Cu:L-Glu | — |
| 21 | —CH₃ | —CH₂COOH | Cu | L-AIa:Cu:L-Asp | — |
| 22 | —H | —CH₂COOH | Cu | Gly:Cu:L-Asp | — |
| 23 | —H | —CH(OH)CH₃ | Cu | Gly:Cu:L-Thr | — |
| 24 | —H | —CH₂CH_{2COOH} | Cu | Gly:Cu:L-Glu | — |
| 25 | —CH₂COOH | —CH(OH)CH₃ | Cu | L-Asp:Cu:L-Thr | — |
| 26 | —CH₂COOH | —CH₂CH₂COOH | Cu | L-Asp:Cu:L-Glu | — |
| 27 | —CH₂CH₂COOH | —CH(OH)CH₃ | Cu | L-Glu:Cu:L-Thr | — |

Example of Imino Acid complexed with Cu (II)

| | | | | |
|---|---|---|---|---|
| 28 | 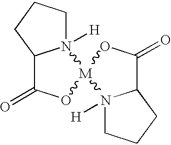 | Cu | L-Pro:Cu:L-Pro | 17.6 |

Examples of alpha-Amino Acids complexed with different metal ions

| | | | | | |
|---|---|---|---|---|---|
| 29 | —CH₂COOH | —CH₂COOH | Ca | L-Asp-Ca-L-Asp | — |
| 30 | —CH₂COOH | —CH₂COOH | Co | L-Asp-Co-L-Asp | |
| 31 | —CH₂COOH | —CH₂COOH | Mg | L-Asp-Mg-L-Asp | |
| 32 | —CH₂COOH | —CH₂COOH | Zn | L-Asp-Zn-L-Asp | |

*compound number
—not tested

TABLE III
Example of a dipeptide complexed with Cu (II)

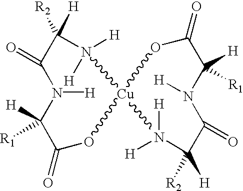

| | | | | Complexes |
|---|---|---|---|---|
| | | | Metal | AA1-AA2:Cu: |
| No.* | R1-R2 | R1-R2 | (M) | AA1-AA2 |
| 33 | R1: | R1: | Cu | (Glu—Trp)₂-Cu |
| | 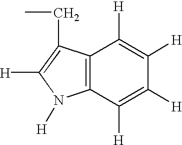 | 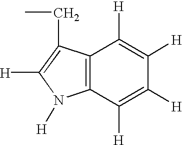 | | |
| | R2: —CH₂CH₂COOH | R2: —CH₂CH₂COOH | | |

*compound number

TABLE IV
Example of a creatine complexed with Cu (II)

| | | Metal | |
|---|---|---|---|
| No* | Structure | (M) | Complexes |
| 34 | 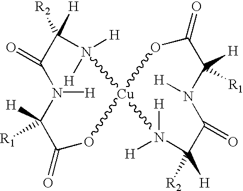 | Cu | (Creatine)₂-Cu |

*compound number

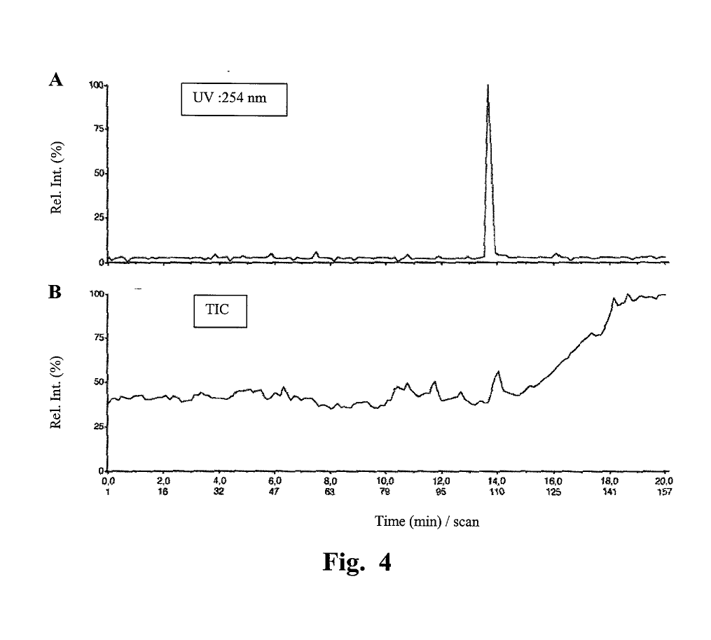

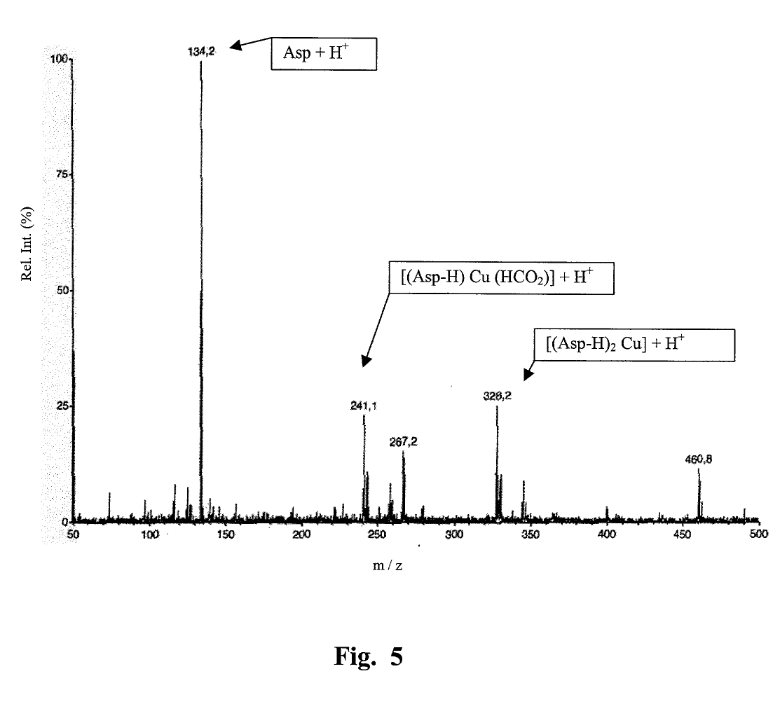

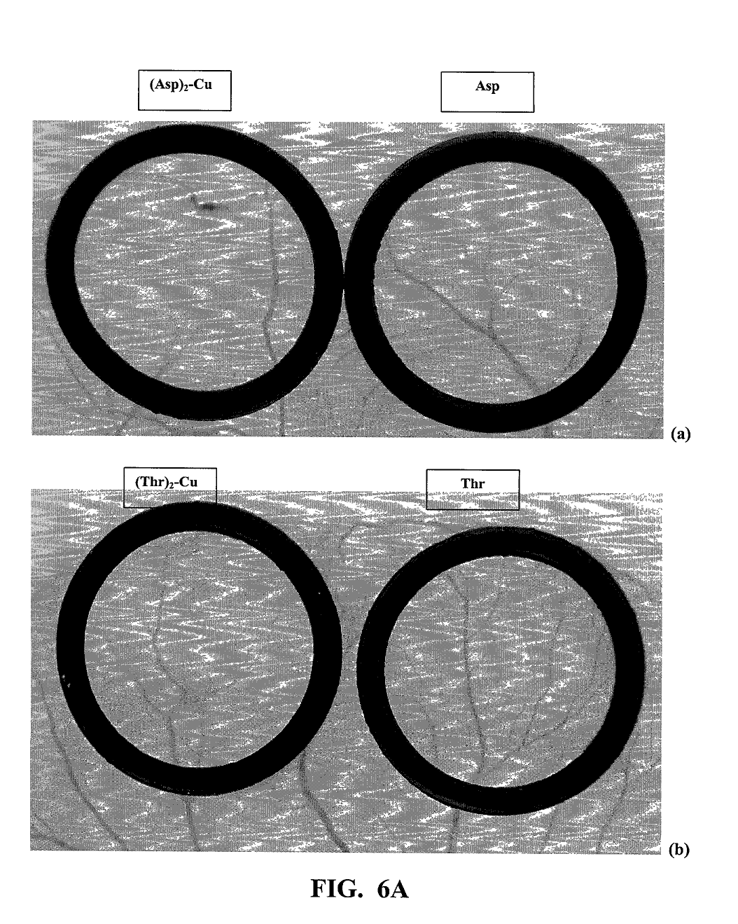

A preferred method for preparing a ligand-metal complex according to the present invention, includes complexing ligands such as natural alpha-amino acids, alpha-imino acids, amino acid analogs, and dipeptides with divalent metals by dissolving water soluble divalent metal salts, for example copper chloride, in water at a molar ratio ranging from 2 to 20 moles of ligand to one mole of divalent metal salt. The pH is then increased to pH 7.0 with ammonium hydroxide. The presence of the ligand-metal complex was verified by Liquid Chromatography-Mass Spectrometry analysis. This procedure was first used to synthesize the (AsP)₂-CU (Compound No. 6 or AE-994) as illustrated in FIGS. 4 and 5. The synthetic compound shows the same chromatographic profile as the AE-994 (compare profile of FIGS. 2 and 3). One of ordinary skill in the art will recognize that other known methods for making such complexes could also be used.

This approach does not allow for the discrimination between a cis or trans conformation or from a planar or tetrahedral configuration of the complex. Moreover, the formation of complexes from the interaction between a reactive residue of an R group of the amino acids and the divalent metal salt cannot be excluded.

Biological Activity of the Complexes

### EXAMPLE 1

### Effect of Test Samples on Endothelial Cell Proliferation and EVT

In order to evaluate the antiangiogenic activity of the compounds prepared according to the processes of the present invention, the endothelial cell proliferation (Table V) and EVT (Table VI) assays were used.

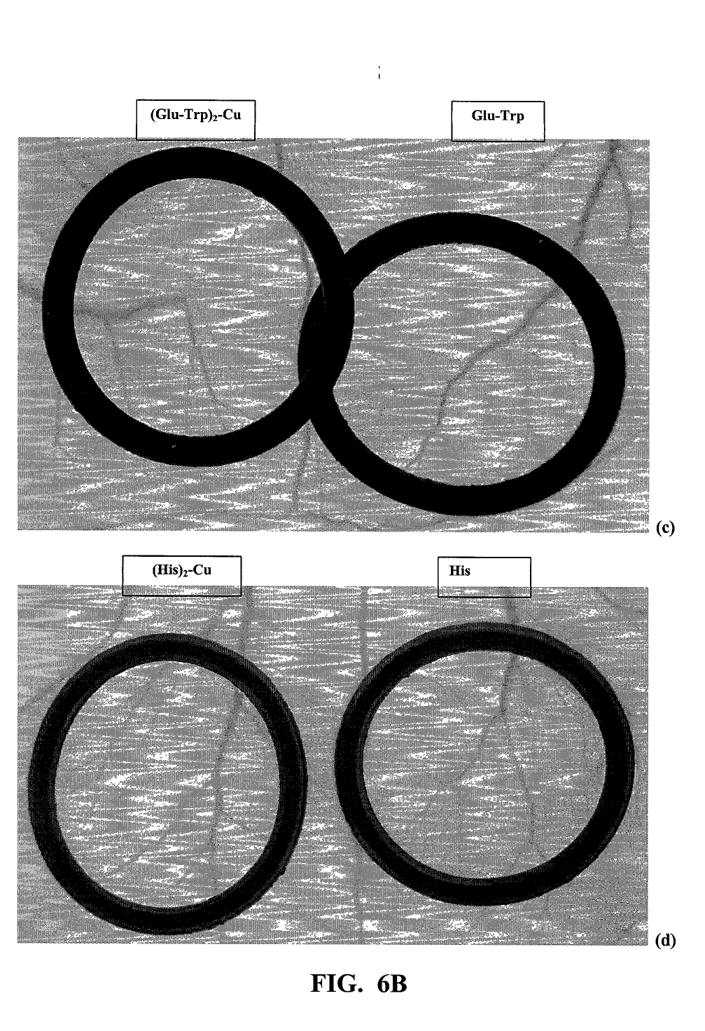

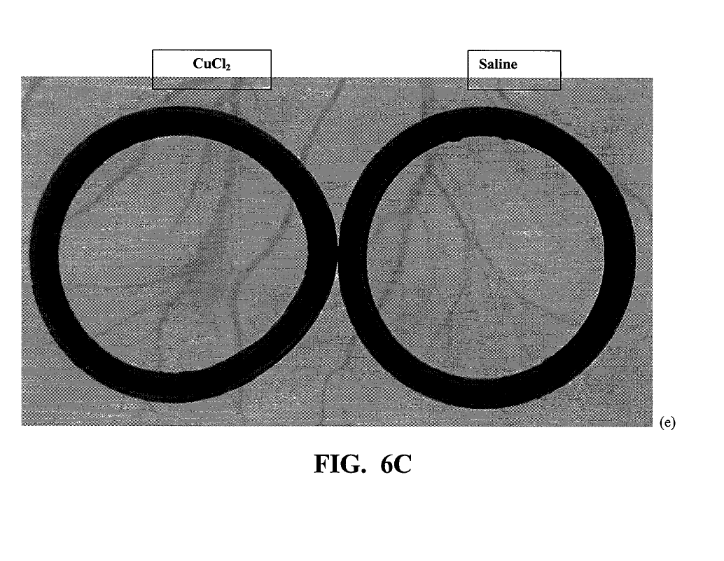

According to the endothelial cell proliferation assay, the relative effectiveness of two compounds is compared by reference to the EC₅₀ thereof. The EC₅₀ is the concentration of compound at which the number of endothelial cells is reduced by 50% as compared to control. The efficacy of the test samples in EVT is established by the percent of eggs with altered angiogenesis at a given inhibitor dose of 0.6 mg of copper equivalent (see FIG. 6 for examples). A comparison of the antiangiogenic activity of the compounds of the present invention with divalent metal salts (CuCl₂, MgCl₂, ZnCl₂, CoCl₂) and uncoupled alpha-amino acids, alpha-imino acids, dipeptides, and nitrogenated molecules comprising a carboxylic acid group, such as creatine, is provided to illustrate that the formation of the amino acid-copper complex gives rise to complexes having antiangiogenic activity.

TABLE V
Endothelial cell proliferation

| Product | EC proliferation | Product | EC proliferation |
|---|---|---|---|
| number | (EC₅₀ mM)) | number | (EC₅₀ mM)) |
| 1 | 0.27 | 21 | 0.32 |
| 2 | 0.28 | 22 | 0.41 |
| 3 | 0.34 | 23 | 0.48 |
| 4 | 0.35 | 24 | 0.33 |
| 5 | 0.30 | 25 | 0.29 |
| 6 | 0.29 | 26 | 0.25 |
| 7 | 0.35 | 27 | 0.40 |
| 8 | 0.32 | 28 | 0.28 |
| 9 | 0.35 | 29 | NA |
| 10 | 0.90 | 30 | 0.27 |
| 11 | 0.76 | 31 | NA |
| 12 | 0.48 | 32 | 0.18 |
| 13 | 0.44 | 33 | 0.23 |
| 14 | 0.37 | 34 | 0.28 |
| 15 | 0.35 | Cu⁺⁺, Ca⁺⁺, Mg⁺⁺ | NA |
| 16 | 0.30 | Zn++ | 0.19 |
| 17 | NA | Co++ | 0.27 |
| 18 | 0.29 | Free AAs | NA |
| 19 | 0.36 | Free dipeptide | NA |
| 20 | 0.28 | Free creatine | NA |

NA (non-active): less than 50% at 1.25 mM copper equivalent, cannot calculate the EC_{50.} Free: means not complexed.

Structure-activity analysis of these compounds indicated that the (His)₂-Cu and (Glu)₂-Cu complexes (compound Nos. 10 and 11), do not appear to form active complexes with copper (II). Histidine and glutamine are two amino acids having a basic moiety in their R residue. The EC₅₀ of compounds 10 and 11 on endothelial cells was significantly higher than 0.50 mM, and each showed very low activity in EVT assay (less then 20%). In contrast, the other amino acid complexes showed important anti-proliferative activity on endothelial cells, with EC₅₀ values ranging from 0.18 to 0.48 mM. These results were confirmed with EVT assay, except for (Ser)₂-Cu complex (compound No. 3) which was inactive in the EVT model. Interestingly, (Thr)₂-Cu complex (compound No. 16), is active. Further, it is possible to obtain active complexes using D-alpha-amino acids instead of L-alpha-amino acids. For example, (D-Ala)₂-Cu, (D-Asp)₂-Cu, (D-Glu)₂-Cu and (Thr)₂-Cu complexes (compounds Nos. 13, 14, 15 and 16) were active in the endothelial cell proliferation assay. In contrast, (D-His)₂-Cu (compound No. 17), a D-histidine-copper complex, was as inactive as the L-form, (L-His)₂-Cu (compound No. 10) in these two assays. Important activity was also observed with complexes made with two different amino acids (where R1 is different than R2). It is interesting to mention that (Pro)₂-Cu (compound No. 28), a proline-copper complex that included proline, an imino-acid, that has a secondary amino function, formed an active complex. Interestingly, creatine-copper complex ((creatine)₂-Cu (compound No. 34)) shows excellent activity on endothelial cell proliferation. This naturally occurring nitrogenated compound was used as an example since high concentrations of creatine are found in cartilage extract. Most interestingly, the formation of a di-peptide complex with copper (Glu-Trp)₂-Cu (compound No. 33) generated one of the most active bioproducts, showing an EC₅₀ of 0.23 mM and 60% inhibition in EVT assay. The equivalent free di-peptide was almost inactive in both assays. These results are in direct contrast with the prior art, where it was declared that such free dipeptide is an inhibitor of angiogenesis in an ex vivo assay wherein the angiogenesis was stimulated with two angiogenic cytokines (VEGF and bFGF). Finally, amino acids complexed with other divalent metals such as Co⁺⁺, Mg⁺⁺, Ca⁺⁺, and Zn⁺⁺(compounds Nos. 29 through 32), appeared to be mostly inactive in vitro, as there was no difference between the activity of the free divalent metal and its corresponding amino-acid-divalent metal complex.

TABLE VI
EVT

| Product number | EVT (% of inhibition)* |
|---|---|
| 1 | ++ |
| 2 | ++ |
| 3 | 0 |
| 4 | + |
| 5 | ++ |
| 6 | + |
| 7 | ++ |
| 8 | + |
| 9 | ++ |
| 10 | 0 |
| 11 | 0 |
| 33 | ++ |
| Free divalent metals | 0 |
| Free AAs | 0 |
| Free dipeptide | 0 |

*activity at 0.6 mg equivalent Copper
0 non-active (less than 25% of inhibition);
+ between 25-50% of inhibition;
++ more than 50% of inhibition;
Free means not complexed.

### EXAMPLE 2

### Proliferation Effect on Non-endothelial Cell Lines

To establish the specificity of action of the compounds of the present invention, their effect on the proliferation of cell types that are different than endothelial cells was tested. Four test samples, compound numbers 5, 6, 10, and 33, were used to represent the present invention. Compound number 5 ((threonine)₂-Cu) and compound number 6 ((aspartate)₂-Cu) correspond to two active amino acid-copper complexes. Compound number 10 is a (histidine)₂-Cu complex, and compound number 33 is a dipeptide-copper complex ((Glu-Trp)₂-Cu.

As represented in Table VII, the test samples did not show important activity on the proliferation of fibroblast and normal striated muscle cells nor on C6 glioblastoma cells. Free amino acids, dipeptide, or copper salts did not show any activity either. These results indicate that the active products of the present invention are selective for endothelial cells, further supporting the antiangiogenic activity of such compounds.

TABLE VII
Cell proliferation

| | Fibroblasts | Striated muscle | C6 glioblastoma |
|---|---|---|---|
| Products | (EC₅₀ (mM)) | (EC₅₀ (mM)) | (EC₅₀ (mM)) |
| 5 | 1.34 | NA | NA |
| 6 | 1.15 | NA | NA |
| 10 | NA | NA | NA |
| 33 | 0.66 | 0.47 | NA |
| Free Asp, | NA | NA | NA |
| Thr, His (2.5 | | | |
| mM) | | | |
| CuCl₂ | NA | NA | NA |
| (1.25 mM) | | | |
| Free Glu-Trp | NA | NA | NA |
| (2.5 mM) | | | |

NA; non-active
Free means not complexed

### EXAMPLE 3

### Endothelial Cell Migration and Tubulogenesis

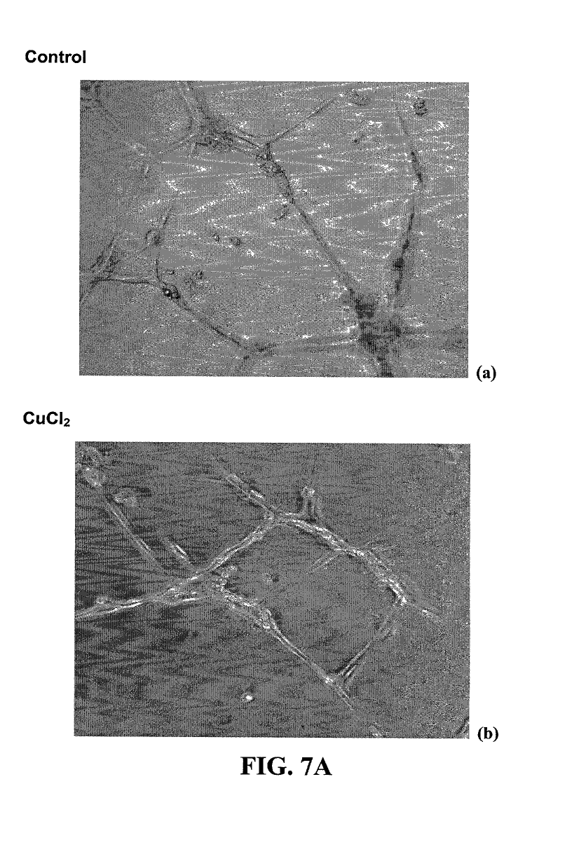

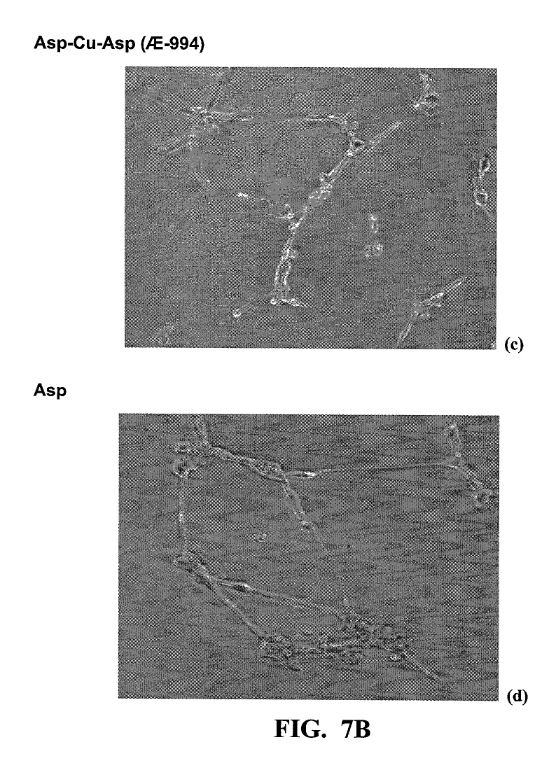

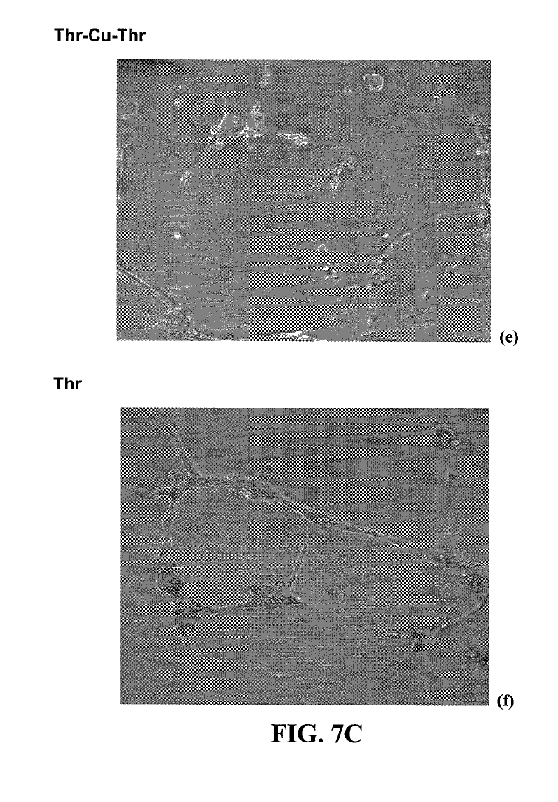

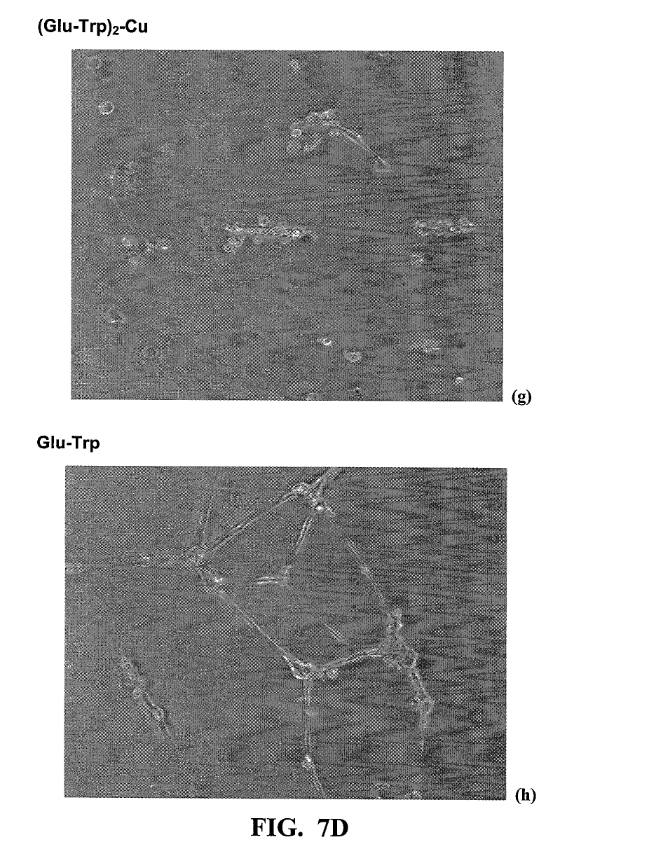

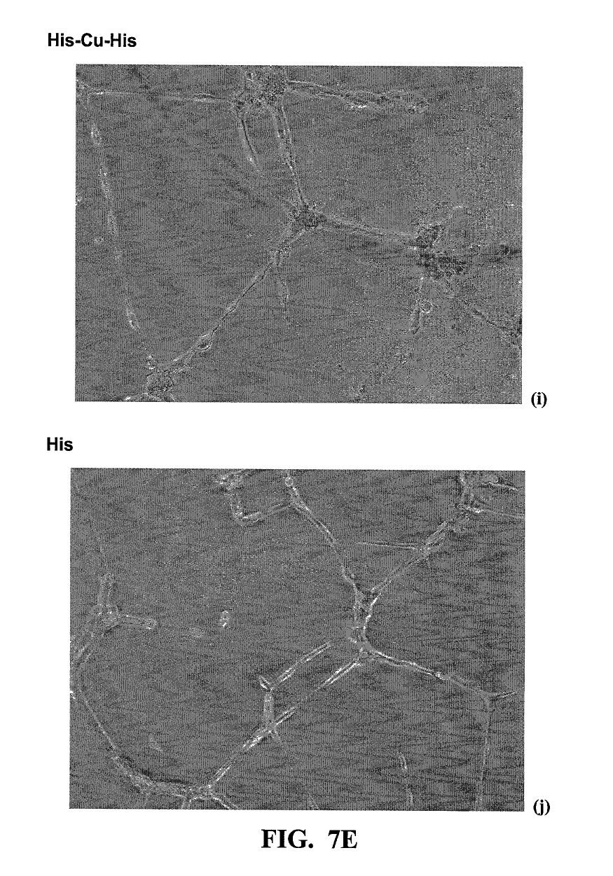

To further support the data on angiogenesis obtained with endothelial cell proliferation and EVT, the activity of test samples on endothelial cell differentiation was determined with the migration and tubulogenesis assays (see FIG. 7). Four test samples, compound numbers 5, 6, 10, and 33, were used to represent the present invention. Compound number 5 ((threonine)₂-Cu) and compound number 6 ((aspartate)₂-Cu) correspond to two active amino acid-copper complexes. Compound number 10 is a (histidine)₂-Cu) complex, and compound number 33 is a dipeptide-copper complex ((Glu-Trp)2-Cu).

As presented in Table VIII, compound numbers 5, 6, and 33 show important activity on cell migration and tubulogenesis, the dipeptide-copper complex being the most active. In contrast, (His)₂-Cu complex (compound No. 10) (the histidine-copper complex), free amino acids, free dipeptide, or copper salts did not show any activity. These results indicated that the active products found in the present invention can also control angiogenesis by inhibiting endothelial cell migration and differentiation.

TABLE VIII
Cell migration and tubulogenesis

| | Cell Migration | |
|---|---|---|
| Products | (% inhibition) | Tubulogenesis* |
| 5 | 90 | ++ |
| 6 | 82 | + |
| 10 | 65 | 0 |
| 33 | 96 | +++ |
| Free Asp, Thr, | NA | 0 |
| His | | |
| (2.5 mM) | | |
| CuCl₂ | 6 | 0 |
| (1.25 mM) | | |
| Free Glu-Trp | 10 | 0 |
| (2.5 mM) | | |

0: Numerous capillary-like tube forming pentagonal network;
+: Decrease of capillary-like tubes
++: Dramatic decrease of capillary-like tubes;
+++: Absence of capillary-like tubes

### EXAMPLE 4

### Angiogenesis Assay with Matrigel™

To establish if the compounds of the present invention are useful for the treatment of disorders related to angiogenesis dysfunction, the antiangiogenic effect of four test samples was determined using an in vivo Matrigel™ assay. Compound numbers 5, 6, 7 and 10 correspond to (threonine)₂-Cu, (aspartate)₂-Cu), (glutamate)₂-Cu and (histidine)₂-Cu), respectively. Results surprisingly indicated that all test samples show antiangiogenic activity in vivo (see Table IX). Moreover, compound 10 was more active than compound 6. These results indicated that these two products can control angiogenesis in vivo and are useful for the treatment of disorders related to a disregulation of angiogenesis. More importantly, it strongly supports the bioavailability of these complexes. Without being bound to any theory, the effect of the compound containing histidine may be due to its high affinity for copper. Compounds having a high affinity for copper may carry more copper to the target cells in angiogenic tissues.

TABLE IX
Effect of test sample on Matrigel ™ in vivo

| Compound number | % inhibition |
|---|---|
| Compound 5 | |
| 1.4 mg/Kg | 47.7 |
| 7.0 mg/Kg | 59.4 |
| Compound 6 | |
| 1.4 mg/Kg | 22.6 |
| 2.5 mg/Kg | 42.0 |
| 7.0 mg/Kg | 56.8 |
| Compound 7 | |
| 1.4 mg/kg | 42.6 |
| 7.0 mg/Kg | 71.2 |
| Compound 10 | |
| 7.0 mg/Kg | 63.4 |
| CuCl₂ (7.0 mg/Kg) | 24.2 |
| Free thr, asp, glu, hist | <15% |

### EXAMPLE 5

### Antimetastatic Activity in vivo

To establish if the compounds of the present invention are useful for the treatment of disorders related to angiogenesis dysfunction, such as metastatic formation in cancer, LLC animal models were used. Three test samples, compound numbers 6, 10 and 33 were used. Compounds number 6 and 10 correspond to (aspartate)₂-Cu) and (histidine)₂-Cu), respectively. Compound number 33 corresponds to the dipeptide complex (Glu-Trp)₂-Cu.

As represented in Table X, the results surprisingly indicated that all test samples show anti-metastatic activity in vivo. Interestingly, the di-peptide(Glu-Trp)₂-Cu complex (compound No. 33) was more active than the free di-peptide. These results indicated that the products of this invention may all be useful for the treatment of angiogenesis-dependent disorders which includes cancer. Even if those compounds were not active in vitro, they were active as anti-angiogenics when used in vivo. Applicants do not therefore disqualify any of the tested analogs as anti-angiogenic therapeutics. From the above results, it is further apparent that at least eight amino acids hark cartilage fraction (SPE-Diol-E-2) will constitute an anti-angiogenic compound, as well as the creatine-copper complex.

TABLE X
LLC

| | Antimetastatic activity |
|---|---|
| | (% inhibition) (at 2.5 mg/kg |
| Product number | of Cu-complex) |
| 6 | 26.9 |
| 10 | 63.0 |
| 33 | 44 |
| CuCl₂ | NA |
| Free di-peptide(glu- | 22.8 |
| trp) | |
| Free Amino acid | NA |

### EXAMPLE 6

### Biodistribution of Test Samples

To establish if the compounds of the present invention can be used via diverse routes of administration, pharmacokinetic analysis was performed on test samples administered by 3 different routes. The test samples were given to mice by oral gavage, by injection intravenous, or intraperitoneal. The results indicated that amino acid-copper complexes can reach blood vessel circulation by each of the three routes tested. This indicates that the present invention may be useful to transport copper to a desired location within the body, wherein the complex is captured by target cells.

### EXAMPLE 7

### Definition of Analogs

From the above results, the compounds of this invention have the following general formula:

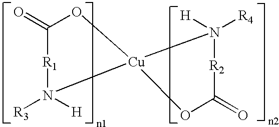

wherein:

n1 and n2 represent units 1 and 2

R1 and R2 may be independently selected from:

RN Substituted alkyl*

RN Substituted amino alkyl

RN Substituted alkyl amide

RN Substituted alkoxy alkyl

RN Substituted alkyl ester, or

RN Substituted alkyl ketone

R3 and R4 may be independently selected from:

Hydrogen

Alkyl

Alkyloic acid

Alkyl amide

Amino alkyl

Alkyl ketone

Alkyl Aldehyde

Alkyl ester

alkoxy alkyl

Halo alkyl

Heterocycles and RNsubstituted heterocycles

Imidazoles and RNsubstituted imidazoles

Aryls and RN substituted aryls, or

Cyclo alkyls and RNsubstituted cyclo alkyls

the RN substituent may be selected from:

Hydrogen

Alkyl

Alcohol

Alkyl alcohol

Carboxylic acid

Alkyloic acid

Amide

Alkyl amide;

Ketone

Alkyl ketone

Imine

Alkyl imine

Thiol

Thiolalkyl

Nitro

Nitro alkyl

Azide

Azido alkyl

Nitrile

Alkyl nitrile

Halide

Halo alkyl

Aldehyde

Alkyl Aldehyde

Ester

Alkyl ester

Ether, or

Alkoxy alkyl

R3 and R4 may cyclisize with R1 or R2 to form heterocycles, and

n1 and n2 may each independently be an amino acid or a dipeptide.

alkyl includes alkane, alkene and alkyne.

By “complex”, is meant two units n₁ and n₂, complexed with copper. Units n₁ and n₂ include amino acids, dipeptides, precursor molecules and any analog thereof, provided that such analog is capable of being complexed with copper and captured by a target angiogenic tissue.

By “analog” is meant any modified amino acid or dipeptide. The modification can be introduced naturally or synthetically. Examples of natural modifications include oxidation, reduction, methylation, hydroxylation and conjugation. Creatine is an example of an analog naturally derived from an amino acid and is itself subject to derivatization. Carnosine is also a naturally occurring dipeptide that may be methylated to form anserine. Ornithine is further a naturally occurring arginine derivative. A last non-limiting example of an analog is sarcosine, which is a natural N-methyl glycine. Examples of synthetic modifications include those made to amino acids or peptides to modify the fluidity of conformation, the lipophilicity or to inhibit the degradation by peptidases. Examples of such modified amino acids include:

acetyl lysine,

acetyl tyrosine,

ε-amino hexanoyl lysyl,

p-(2-thienyl)-alanyl,

β-naphtyl-alanine,

1,2,3,4-tetrahydroisoquinoline-3carboxyl, and

3aS,7aS-octahydro-indol-2-carboxyl.

By “creatine derivative”, is meant a creatine that has been modified following the description found in U.S. Pat. No. 6,114,379, the entire disclosure of which is hereby incorporated herein by reference.

### EXAMPLE 8

### Compositions and Uses

The present compounds and compositions can be used for the treatment of angiogenesis-dependent diseases by inhibiting angiogenesis in a target tissue.

By “angiogenic target tissue” is meant a tissue formed of cells that are undergoing, are susceptible to undergo, or susceptible to participation in angiogenesis. This tissue may comprise tumor cells or other cells that provoke blood vessel-forming cells to migrate and proliferate towards them. This tissue may also comprise the blood vessel-forming cells themselves, especially endothelial cells. No matter which cell exactly responds to the present compounds and how it responds, the above results indicate that angiogenesis is inhibited. Some of the compounds appear capable of directly inhibiting endothelial cell proliferation. Others do not, but yet are still anti-angiogenic. Thus, there may be also an indirect action of the present complexes on endothelial cells that is mediated by another type of cell or factor.

The compounds of the present invention may be used in the presence of a pharmaceutically acceptable vehicle. These constitute the first basic compositions. The complexes may also be combined in any possible mixture. Such mixtures constitute a second type of basic compositions.

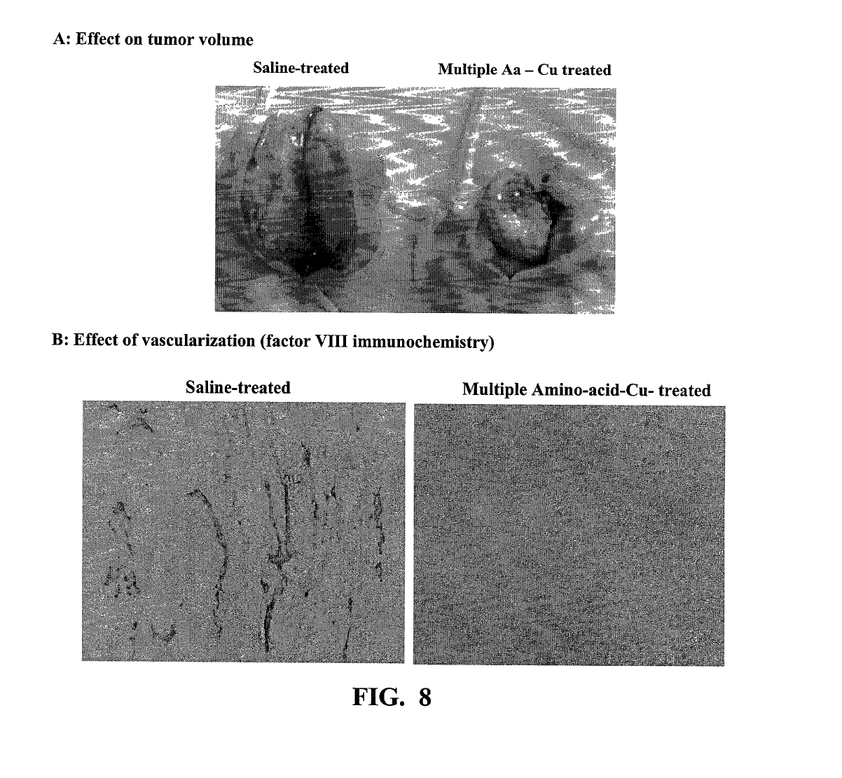

An example of such a second type of basic composition shows higher antiangiogenic activity than single amino-acid-copper complexes. For example, a composition containing a mixture of eight different amino acids (Thr, Asp, Glu, Gly, Ala, Pro, Gln, Ser) complexed with Cu⁺⁺ at 1.4 mg/Kg (equivalent of copper concentration) inhibits angiogenesis, in vivo, by 71%, upon intraperitoneal administration in saline. This mixture corresponds to the SPE-Diol-E-2 fraction. Such antiangiogenic property appears to be related to strong antitumor activity, as demonstrated in the C6 glioblastoma model. In the C6 glioblastoma model, a significant decrease of the tumor volume (69% at 7.0 mg/Kg) and an almost complete inhibition of tumor vessels was observed (FIG. 8). Moreover, the same composition of matter shows a strong antiangiogenic activity as shown by an EC₅₀ of 0.23 mM on endothelial cell proliferation and a significant decrease of blood vessel formation in the EVT assay.

The complexes of the present invention may further be combined with any other therapeutic agent, namely a therapeutic agent that complements the therapeutic activity of the present complexes. The first category of such other therapeutic agents would include any other anti-angiogenic drug. When the treatment of cancer cells is of concern, the other therapeutic agent could include an anti-tumor agent. An anti-tumor agent includes but is not limited to anti-angiogenics, anti-neoplastics (chemiotherapeutics and radiotherapeutics), immunotherapeutic agents, and anti-collagenolytic agents. Since inflammation, formation of reactive oxygen species and angiogenesis are interrelated events, it is also considered that another therapeutic agent that could be combined with the present complexes could include anti-inflammatory agents, anti-oxidants and anti-angiogenics.

A therapeutic agent that conveniently combines anti-tumor, anti-angiogenic, anti-inflammatory and anti-collagenolytic activities is shark cartilage extract. By combining a shark cartilage extract with any one of the complexes of this invention, an “enriched extract” is provided. Such an “enriched extract” provides a combination that has high anti-angiogenic potency, other therapeutic activities, and is also innocuous.

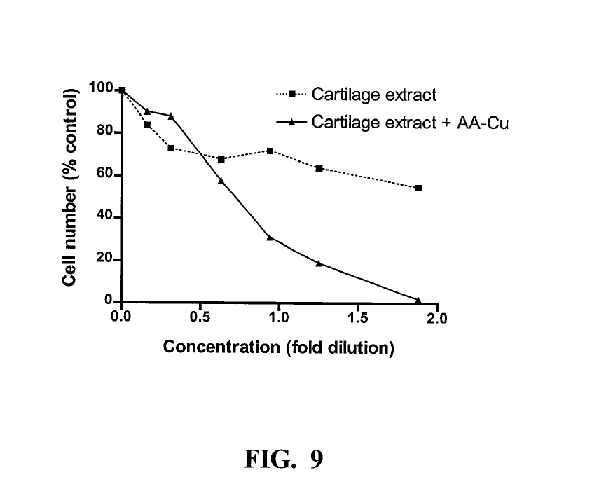

Another such exemplary composition is one comprising multiple-amino-acid-copper complexes and a liquid shark-cartilage extract. 5 mg protein/mL of shark cartilage extract was mixed with the 5 amino acids (Thr, Asp, Glu, Ala, Gly) complexes with copper removed SPE-Diol-E-2 replaced by a fraction of the same shark cartilage extract (0.1 mM of copper equivalent). This mixture was serially diluted and tested against endothelial cells. This mixture constitutes an “enriched extract” which has a higher anti-angiogenic activity (FIG. 9). These examples revealed that compositions comprising amino acid-copper complexes may be useful for the treatment of disorders related to angiogenesis dysfunction.

The examples could also be combined with any of the above other therapeutic agents that are different from shark cartilage extract. For example, International Publication No. WO98/40088, discloses compositions comprising anti-neoplastic agents and a shark cartilage extract that show an increased anti-tumor activity and a protective effect against toxic side effects. Therefore a composition which would combine an anti-neoplastic, a shark cartilage extract, and the complexes of the present invention would still protect the treated subject against the severity of toxic side effects of anti-neoplastics, with the benefit of an increased anti-angiogenic activity.

Anti-oxidants may also be complementary therapeutic agents, particularly for treating angiogenesis-dependent diseases and oxidative diseases. Oxidatives diseases are caused or exacerbated by the production of deleterious oxygen-reactive species (ROS). ROS are known to participate or intiate inflammation and apoptosis.

The “subjects” to be treated comprise any organism, including mammals, wherein angiogenesis occurs and needs to be controlled.

An “angiogenesis-dependent disease” is any disease, condition or disorder, wherein angiogenesis undesirably takes place and needs to be controlled, prevented or inhibited. This includes diseases such as arthritis, psoriasis, and cancer, as well as any other diseases listed in International Publication Nos. WO 95/32722, WO 96/23512 and WO 97/16197; Griffioen A W. Angiogenesis: Potentials for Pharmacologic Intervention in the Treatment of Cancer, Cardiovascular Diseases, and Chronic Inflammation, Pharmacol. Rev. 52:237-68, 2000; Brem S. Angiogenesis and cancer control: from concept to therapeutic trial, Cancer Control. 6:436-458, 1999; Hu G F. Copper stimulates proliferation of human endothelial cells under culture. Cell Biochem 69:326-335, 1998; and Sauder and Thibodeau, Angiogenesis in Dermatology, Curr. Probl. Dermatol, May/June 2001, in press.

To treat such angiogenesis-dependent diseases, an effective dose of the present complexes is used. The “effective dose” is that dose which has an anti-angiogenic effect. Preferably, an effective dose is between 0.1 to 10 mg of copper equivalent per Kg of body weight, preferably between about 1 and 10 mg/Kg of body weight. The effective therapeutic doses of 5 mg/Kg (equivalent copper) have been administered intraperitoneally in murine models. Such dose achieves a maximal plasmatic concentration ranging from 250 to 400 μmoles per liter. The dose should be selected upon the route of administration, the bioavailability and the aggressiveness of the treatment as well as the metabolic pathways that are particular to the subject.

Pharmaceutical compositions can be in any suitable form adopted to any desired route of administration. Both enteral and parenteral routes of administration are considered to be such desired routes. Compositions may take the form of solutions, suspensions, powders or solubilizable granules, syrups or elixirs, auricular, nasal or ophthalmic drops, tablets, gelatin-coated pills, aerosols, ointments, transdermal applications or suppositories, in dosed presentations containing non-toxic supports, adjuvants and excipients. The injections can, for example, be intravenous, intramuscular, subcutaneous, intradermal, intrasternal or intra-articular.

The compositions can also include any other compound that helps preserve or enhance the activity of the complexes. Pharmaceutical formulations comprising buffers, salts, solubilizers, permeation enhancers, surfactants, viscosity enhancers, stabilizers and anti-oxidants are all examples of such compounds known to those of ordinary skill in the art. Such compounds are within the definition of “a pharmaceutically acceptable vehicle”. Each vehicle is chosen upon the route of administration and the desired texture.

### EXAMPLE 9

### Fabrication Process and Starting Material

In the present invention, the cartilage source is not limited to shark cartilage. The process by which the present complexes are obtained are certainly not restricted to one starting with cartilage tissue. Amino acids or dipeptides obtained from commercial sources or from protein hydrolysis can be complexed with copper simply by mixing with a copper salt solution and adjusting the pH to a basic value. When cartilage tissue is the starting proteic material comprising precursors, an acidic treatment is performed with acids like TFA, phosphoric acid, citric acid, acetic acid, formic acid and trichloroacetic acid, which all provide a mild treatment to denature, decompose or decomplex bigger molecules. Heat at temperatures between 37° C. and 100° C. also provide amino acids and dipeptides in low molecular weight fractions complexed with copper. This equivalent treatment reinforces the idea of “denaturing” bigger molecules or hydrolizing the same, which would result in detaching and releasing small molecules from bigger ones.

Other compounds having nitrogenous and carboxylic groups may be synthesized and complexed with divalent metals, to fulfill the goal of the invention. A library of such nitrogenous compounds is described by Cook et al. in U.S. Pat. No. 6,197,965, the entire disclosure of which is hereby incorporated herein by reference. This library can be screened for candidates capable of complexing divalent metals and further tested for their anti-angiogenic, anti-metastatic and anti-tumor properties.

Although the present invention has been described hereinabove by way of preferred embodiments thereof, it can be modified, without departing from the spirit and nature of the subject invention as defined in the appended claims.

What is claimed is:

1. A method for selectively inhibiting angiogenesis which comprises the step of administering to a subject an effective amount of an anti-angiogenic compound consisting essentially of two units complexed to a copper metal ion, wherein said units are independently selected from the group consisting of an amino acid, a dipeptide or an analog thereof which has a carboxyl and an amino group capable of complexing with copper and that targets cells of an angiogenic tissue.

2. A method for obtaining a compound which has an anti-angiogenic activity from a cartilage material which comprises the steps of:
a) extracting said anti-angiogenic activity from cartilage material reduced to solid particles whose size is less than or equal to about 500 μm into an aqueous solution, resulting in a homogenous mixture of said solid particles and a first liquid extract having said activity;
b) separating solid particles from said first liquid extract;
c) fractionating said first liquid extract, to recover a second extract comprising molecules having a molecular weight less than about 500 KDa; and
d) treating said second extract under denaturating conditions of pH or temperature to generate a low molecular weight compound comprising amino acids, dipeptides, analogs thereof, and copper-complexes thereof.

3. The method of claim 2 wherein said pH ranges from about 2 to about 6.

4. The method of claim 2 wherein said temperature ranges from about 37° C. to 100° C.

5. The method of claim 2, further comprising the step of purifying said compound from said neutralized extract.

6. A cartilage extract obtained from the process of claim 2.

7. The cartilage extract of claim 6 wherein said cartilage extract is a shark cartilage extract.

8. An anti-angiogenic compound consisting essentially of two units complexed to a copper metal ion, wherein said units are independently selected from the group consisting of an amino acid, a dipeptide or an analog thereof which has a carboxyl and an amino group capable of complexing with copper and that targets cells of an angiogenic tissue.

9. A compound as defined in claim 8, wherein said amino acid is selected from the group consisting of: threonine, aspartic acid, glutamic acid, glycine, alanine, valine, leucine, isoleucine, arginine, lysine, proline, glutamine, serine and histidine.

10. A compound as defined in claim 8, wherein said dipeptide is glutamyl-tryptophane.

11. A compound as defined in claim 8, wherein said analog is creatine or a creatine-derivative.

12. A composition of matter consisting essentially of an effective amount of a compound as defined in claim 8, and a pharmaceutically acceptable vehicle.

13. A composition of matter as defined in claim 12, wherein said amino acid is selected from the group consisting of threonine, aspartic acid, glutamic acid, glycine, alanine, valine, leucine, isoleucine, arginine, lysine, proline, glutamine, serine, histidine, or any mixture thereof.

14. A composition as defined in claim 12, wherein said dipeptide is glutamyl-tryptophane.

15. A composition as defined in claim 12, wherein said analog is creatine or a creatine-derivative.

16. A composition as defined in claim 12, which further comprises an anti-inflammatory, an anti-tumor agent, an anti-oxidant, or an anti-collagenolytic agent.

17. A composition as defined in claim 12, which further comprises an anti-tumor agent.

18. A composition as defined in claim 17 wherein said anti-tumor agent comprises a shark cartilage extract.

19. A composition as defined in claim 17, wherein said anti-tumor agent comprises a shark cartilage extract and an anti-neoplastic agent.
